(19) United States
(12) Patent Application Publication
(10) Pub. No.: US 2015/0254850 A1
(43) Pub. Date: Sep. 10, 2015

(54) SYSTEM FOR DETECTING BLOOD VESSEL STRUCTURES IN MEDICAL IMAGES

(71) Applicants: Region Nordjylland, Aalborg Sygehus, Aalborg Ø (DK); Aalborg Universitet, Aalborg Ø (DK)

(72) Inventors: Alex Skovsbo Jørgensen, Aalborg (DK); Lasse Riis Østergaard, Klarup (DK); Samuel Emil Schmidt, Aalborg SO (DK); Niels-Henrik Staalsen, Nibe (DK)

(73) Assignee: Aalborg Universitet, Aalborg (GB)

(21) Appl. No.: 14/426,256

(22) PCT Filed: Sep. 6, 2013
(86) PCT No.: PCT/DK2013/050284

§ 371 Date: Mar. 5, 2015

(30) Foreign Application Priority Data
Sep. 7, 2012 (DK) PA 2012 70546

(51) Int. Cl. G06T 7/00; G06T 7/60; A61B 5/00; G06T 3/00

(52) U.S. Cl. CPC G06T 7/0014; G06T 7/0016; G06T 7/0044; G06T 3/0068; G06T 3/0093; G06T 7/602; A61B 5/489; G06T 2207/10004; G06T 2207/20044; G06T 2207/30101; G06T 2207/30104; G06T 2210/41; G06T 2207/20148; G06T 2207/20152

(57) ABSTRACT

The invention relates to method for detecting blood vessel structures in medical images obtained from a medical imaging device, wherein the method comprises determining image parts in a selected medical image, where the image parts are determined by processing intensity values of the image, determining first and second feature values of each of one or more of the image parts, determining one or more feature values of each of one or more of the image parts, where the features values indicate if the image part from which the feature values are determined pictures a desired blood vessel structure, based on the feature values, determine if the selected medical image shows the desired blood vessel structure.

### FIELD OF THE INVENTION

The invention relates to image processing of medical images, in particular to analysing images for detecting blood vessel structures.

### BACKGROUND OF THE INVENTION

In image processing of medical images which images vessel structures in the human body it may be difficult to automatically detect the vessel in the image. The problem arises since the vessel image may not be perfectly imaged and since the image may contain other structures which may be difficult to distinguish from the vessel structure by automatic image processing methods.

WO2012107050 discloses a method for providing quantitative measures of the flow property of a blood vessel. The method is based on analyzing cross-sectional images of a vessel by estimating the area of the lumen of the vessel. The method comprises steps of determining a point contained within the walls of the vessel, determining a closed path which approximates the inner circumference of the wall of the vessel, and determining the area of the closed path when the vessel is most expanding in order to get a measurement of the maximum lumen. This method may enable the clinical personnel to quickly evaluate the flow property e.g. of an inserted bypass vessel and, thereby, conclude if the surgical intervention is successful or if adjustments are required.

### SUMMARY OF THE INVENTION

It may be seen as an object of the present invention to provide a method that improves automatic detection of vessel in medical images or other problems of the prior art.

To better address one or more of these concerns, in a first aspect of the invention a system for detecting blood vessel structures in medical images obtained from a medical imaging device configured to obtain cross sectional views of blood vessels is presented where system comprises a processing unit configured for analysing the medical images by performing the steps:
determining image parts in a selected medical image, where the image parts are determined by processing intensity values of the image,
determining one or more feature values of each of one or more of the image parts, where the features values indicate if the image part from which the feature values are determined pictures a desired blood vessel structure,
based on the feature values, determine if the selected medical image shows the desired blood vessel structure.

The processing unit may be electronic hardware and/or a processor for executing computer program code, where the hardware and/or the computer program is configured for analyzing the images.

The feature values may indicate if the image part from which the feature values are determined pictures a desired blood vessel structure by use of classification method, statistical methods or by use of probability distributions. In general different values of a give feature are associated with different degrees of probabilities that a given value is associated with the finding of a desired vessel structure in an image part.

By analysing a plurality of feature values determined from each of a plurality of image parts in a medical image it may be possible to improve automatic detection of desired vessel structures in medical images.

In an embodiment the feature values are associated with a likelihood/probability that the image part from which the feature values are determined pictures a desired blood vessel structure.

In an embodiment the processing unit is configured for analysing the medical images by determining one or more of the following feature values of each of one or more of the image parts:
a) an intensity standard deviation feature value determined by calculating the standard deviation of intensity values of pixels contained in one of the image parts,
b) a mean intensity feature value determined by calculating the mean value of intensity values of pixels contained in one of the image parts,
c) a compactness feature value determined by calculating the boundary length and the area within the boundary of one of the image parts and comparing the boundary length with the area,
d) a vertical distance feature value determined by calculating a distance between a center value of one of the image parts and a center value of the medical image,
e) a horizontal distance feature value determined by calculating a distance between a center value of one of the image parts and a center value of the medical image,
f) a boundary gradient feature value determined by calculating the first derivative of the intensity values of pixels contained in one of the image parts,
g) an intensity variance feature value determined by calculating the variation of intensity values of pixels contained in one of the image parts, and
h) an aspect ratio feature value determined by calculating a major axis and a minor axis of one of the image parts and comparing the major axis with the minor axis.

In an embodiment the processing unit is configured for analysing the medical images by performing the steps
determining first and second feature values of each of one or more of the image parts,
comparing the first and second feature values with respective first and second probability distributions, where the distributions describe the likelihood that the image part from which the feature values are determined pictures a desired blood vessel structure.
based on the comparison, determine if the selected medical image shows the desired blood vessel structure with a sufficiently high likelihood.

In an embodiment comparing the first and second feature values with respective first and second probability distributions comprises determining respective first and second probability values from the probability distributions corresponding to the feature values.

An embodiment further comprises calculating a sum of the first and second probability values.

An embodiment further comprises using the selected medical image for further image processing or discarding the selected medical image based on the determining if the selected medical image shows the desired blood vessel structure.

In an embodiment the medical images represent a time series of images showing a pulsating blood vessel, wherein the selected image is a first image in the time series of images, and wherein
the geometric center of a finally adapted contour of the desired blood vessel structure in the first image is determined,
the finally adapted contour is used as an initial contour in a subsequent image in the time series of images,
an intensity center in the initial contour is calculated from intensity values of pixels of the subsequent image which are contained within the initial contour of the desired blood vessel structure in the first image,
the initial contour is adapted or displaced so as to minimize the distance between the geometric center and the intensity center.

As second aspect of the invention relates to a method for detecting blood vessel structures in medical images obtained from a medical imaging device configured to obtain cross sectional views of blood vessels, wherein the method comprises
determining image parts in a selected medical image, where the image parts are determined by processing intensity values of the image,
determining first and second feature values of each of one or more of the image parts,
comparing the first and second feature values with respective first and second probability distributions, where the distributions describe the likelihood that the image part from which the feature values are determined pictures a desired blood vessel structure.
based on the comparison, determine if the selected medical image shows the desired blood vessel structure with a sufficiently high likelihood.

### BRIEF DESCRIPTION OF THE DRAWINGS

Embodiments of the invention will be described, by way of example only, with reference to the drawings, in which

FIG. 1 schematically illustrates a medical image 100 picturing a cross sectional view of a blood vessel 101 and the lumen 102 of the blood vessel,

FIG. 2 shows a medical image showing two vessels 101a, 101b and their lumina 102a, 102b,

FIG. 3 shows three graphs for determining a probability distribution,

FIG. 4 shows histograms, and

FIG. 5 and FIG. 6 shows cross sectional views of a blood vessel.

### DETAILED DESCRIPTION OF AN EMBODIMENT

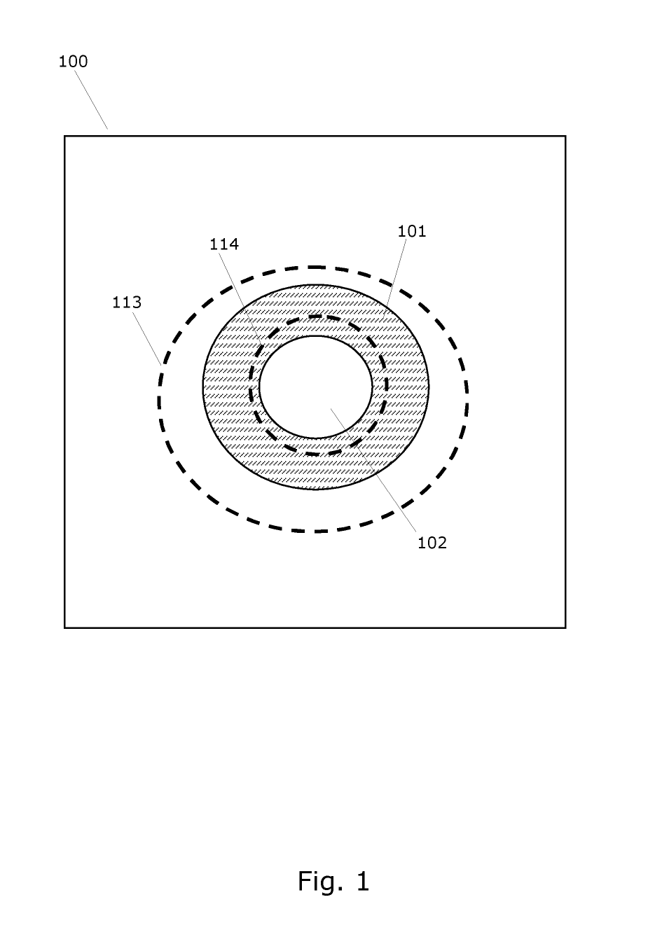

FIG. 1 schematically illustrates a medical image 100 picturing a cross sectional view of a blood vessel 101 and the lumen 102 of the blood vessel.

An embodiment of the present invention relates to a method for detecting blood vessel structures 101 in medical images 100 obtained from a medical imaging device, e.g. an ultrasound imaging device or a magnetic resonance imaging device. In order to be able to analyse the blood vessel structures 101, e.g. calculating the area of the lumen 102, it is necessary to create an analytical or mathematical model of the blood vessel structures, e.g. a model describing the boundary between the vessel tissue 101 and the lumen 102. Accordingly, in an embodiment the method for detecting blood vessel structures 101 further comprises methods for modeling the blood vessel structures 101.

The process of detecting blood vessel structures in medical images comprises one or more of the following steps:
1) determining image parts in a first medical image 100,
2) determining feature values of each of one or more of the image parts,
3) comparing the feature values with probability distributions in order to determine if the selected medical image shows the desired blood vessel structure with a sufficiently high likelihood,
4) creating an adaptable closed circular contour for modeling e.g. the boundary between the vessel tissue 101 and the lumen 102,
5) deforming the adaptable closed circular contour towards the boundary or wall between the vessel tissue 101 and the lumen 102 to obtain a mathematical model or description of the blood vessel structure,
6) using the finally adapted contour from step 5 as an initial contour of the boundary between the vessel tissue and the lumen in an image part in a subsequent second medical image 100.

The method for detecting blood vessel structures in medical images may be used for analysing still images, but is particularly suited for analysing a time series of medical image frames. Such medical image video may be obtained by the medical imaging device for determining area-values of the blood vessel lumen as function of position along a blood vessel by moving the medical imaging device along the vessel while images are being recorded.

Steps 1), 4) and 5) are described in detail in patent publication WO2012/107050 which is hereby incorporated by reference.

Steps 1)-6) are described in more detail below.

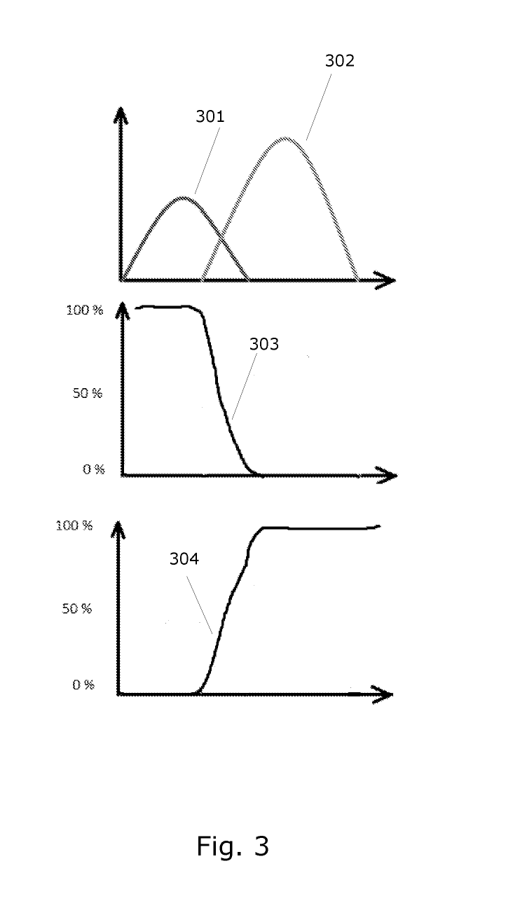

Thus, the determination of image parts in a first medical image 100 according to step 1) may be performed by use of the canny method for determining edges in the image as described by steps 1-6 on pages 12-13 in WO2012/107050. The result of the canny method may be the inner edge 304 of the blood vessel as shown in FIG. 3 in WO2012/107050. FIG. 1 illustrates an image part 114 being delimited from other parts of the image 100 by edge shown as the broken closed line. The edge of the image part 114 may have been found by the Canny method or other suitable edge detection method. The image part 114 pictures the lumen 102 of the vessel 101. Other image parts such as image part 113 which pictures a cross sectional view of the tissue of the blood vessel may be found according to step 1.

In a similar solution, the determination of image parts in a first medical image 100 according to step 1) may be performed by use of watershed segmentation on the image 100 followed by an adaptive thresholding. The watershed segmentation is used to extract vessel candidate regions where a vessel could be present. It is performed on the image 100 preprocessed with a Gaussian low pass filter to obtain gross anatomical details only. As the watershed segmentation often overestimates the vessel lumen 102 the adaptive thresholding is used to extract a possible vessel lumen region 102. The adaptive threshold is performed on the image 100 filtered with a median filter (kernel=30×30) followed by a Gaussian low pass filter to obtain a uniform vessel lumen region. The threshold may be set to 20% of the dynamic range inside the watershed region added to the minimum intensity value in the same region. Pixels below the threshold are defined as possible lumen pixels.

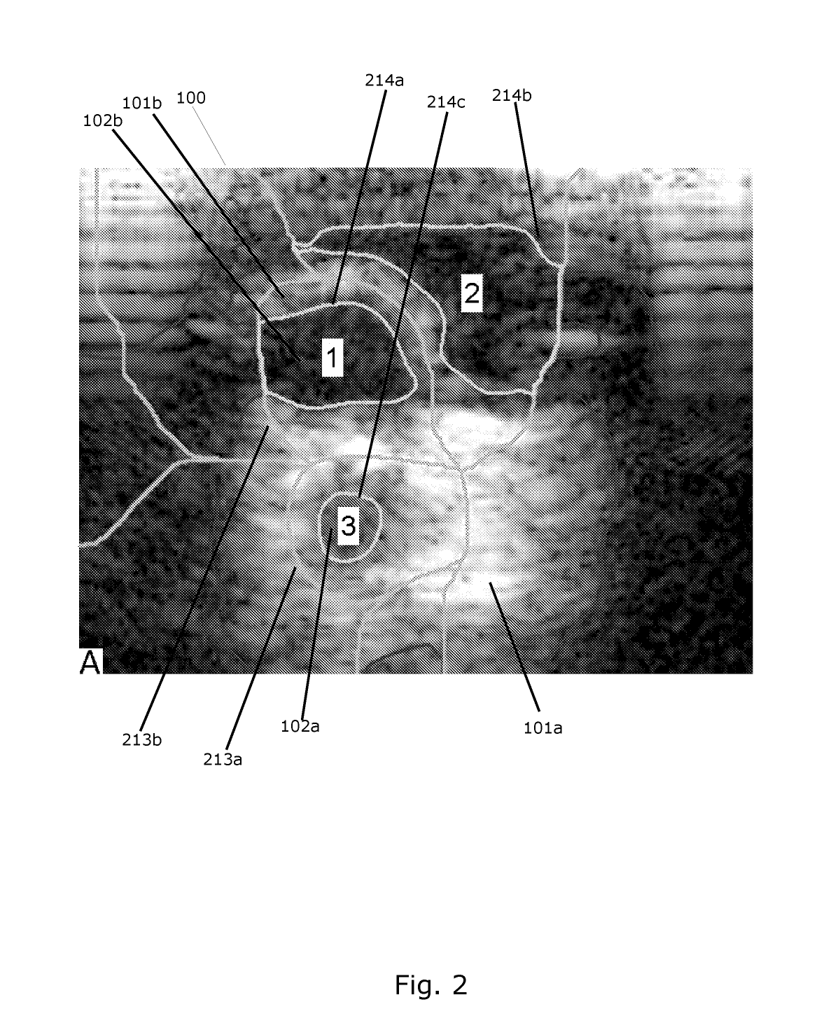

FIG. 2 shows a medical image showing two vessels 101a, 101b and their lumina 102a, 102b. Image parts 213a and 213b contain cross sectional views of the vessel tissue of vessels 101a and 101b, respectively. Image parts 214a, 214b and 214c contain cross sectional views of what could be the lumina 1, 2 and 3, respectively, of blood vessels. The borders of image parts 213a, 213b may have been determined e.g. by the watershed segmentation or by the Canny method. The borders 214a-214c may have been determined by the adaptive the thresholding method or by the Canny method.

In general the Canny method, the watershed segmentation and the adaptive thresholding method or other method for determining the image parts 113, 114, 213a, 213b, 214a, 214b are based on processing intensity values of the image. That is, the boundary between vessel tissue 101 shown as high intensity pixels and the lumen 102 shown as low intensity pixels can be determined by finding those pixels where the intensity changes most rapidly over neighbour pixels.

From FIG. 2 it is clear that image part 214b does not represent an image of a lumen, only image parts 214a and 214b contain images of lumina 1 and 3 respectively.

Steps 2) and 3) are capable of determining which of image parts 213a-b and 214a-c actually contains images of a vessel lumen 102 or other desired blood vessel structures.

In step 2) features values, such as first and second feature values, of each of one or more of the image parts 113, 114, 213a, 213b, 214a, 214b are determined. The feature values may be determined by different image processing methods in order to transfer characteristics of the image parts into different feature values. Different methods for determining feature values are described in detail below.

An intensity standard deviation feature value may be determined by calculating the standard deviation of intensity values of pixels contained in the image part. This feature provides a measure of the contrast within the image part. This feature is therefore suited for detecting presence of blood vessels for image parts 113, 213a, 213b which contains both the vessel tissue 101 and the vessel lumen 102. That is, the vessel has a relatively high contrast due the high intensity pixel values of the tissue part 101 and the low intensity pixel values of the lumen part 102. Non-vessel image parts which does not contain a cross sectional view of the vessel tissue 101 and the lumen 102 tend to have smaller intensity standard deviation feature values than image parts which contain a cross sectional view of the vessel tissue 101 and the lumen 102.

A mean intensity feature value may be determined by calculating the mean value of intensity values of pixels contained the image parts. Since image parts 114, 214a, 214b and 214c primarily contain low intensity pixels, the mean intensity feature value is suited for detecting such image parts showing a vessel lumen 102 since the vessel lumen 102 primarily contains low intensity pixels.

A compactness feature value may be determined by calculating the boundary length and the area within the boundary (e.g. the broken line of image part 114) of one of the image parts and by comparing the boundary length with the area, e.g. by calculating the ratio of the squared boundary length and the area. The compactness feature value is suited for characterizing how circular an image part is. Since vessels 101 and vessel lumina 102 have a circular shape the compactness feature value is suited for detecting image parts containing a vessel structure. Disc-shaped regions generate low compactness feature values compared to image parts with a non-circular shape.

A vertical distance feature value may be determined by calculating a distance between a center value of one of the image parts and a center value of the medical image. This feature is suited for detecting image parts containing vessel structures 101, 102 since operator of the imaging device normally will position the vessels in the middle of the image. This feature will therefor give less weight to regions located in the top and bottom of the image compared to regions located in the center of the image.

The vertical distance feature value may be determined as a signed distance feature value as there can be a distinct difference between regions detected in the top and the bottom of the image.

A horizontal distance feature value may be determined by calculating a distance between a center value of one of the image parts and a center value of the medical image. This feature will give less weight to image parts located to the left and right side of the image compared to regions located in the center of the image and is therefore suited for detecting vessel structures 101, 102 for the same reason as the vertical distance feature.

A boundary gradient feature value may be determined by calculating the first derivative of the intensity values of pixels contained in one of the image parts, e.g. by calculating the difference between intensity values between two neighbor pixels and summing the differences over the pixels of the image part. The boundary gradient feature is suited for characterizing how much the intensity content changes in an image and is therefore suited for characterizing edges in an image as significant edges have a high gradient value. The gradient feature value, which may be calculated as a mean value, is therefore suited for detecting if the boundary of an image part is located in a significant gradient.

An intensity variance feature value may be determined by calculating the variation of intensity values of pixels contained in one of the image parts. The intensity variance is suited as a measure of homogeneity in a region and it is therefore suited for detecting vessel structures since the variance of intensity pixel values of the vessel lumen region is relatively homogeneous. The intensity variance will be low in more homogeneous regions.

An aspect ratio feature value may be determined by calculating a major axis and a minor axis of one of the image parts and by comparing the major axis with the minor axis, e.g. by calculating the ratio. The aspect ratio feature is suited for describing the proportional relationship between the width and length of the adaptive threshold region by calculating the major- and minor axis of the region. This feature is therefore suited for detecting vessel structures which have a relatively circular shape, i.e. which have approximately the same length of the major and minor axes.

In step 3) the feature values (i.e. any of the above described feature values) are compared with associated probability distributions. E.g. the first and second feature values may be compared with respective first and second probability distributions. The probability distributions describe the likelihood that the image part from which a given feature value is determined pictures or shows a desired blood vessel structure such as a vessel lumen. Based on this comparison, it is determined if the medical image—selected as the first image—shows the desired blood vessel structure with a sufficiently high likelihood. If the likelihood is sufficiently high, image processing on the selected image is continued according to steps 4)-6).

When more than one feature value is determined for a given image part, the determination of a vessel likelihood may comprise calculating a sum of the probability values. The sum may further be calculated as a weighted sum in order to give more or less weight to some of the feature values.

The probability distributions can be determined from training images.

FIG. 3 shows three graphs where the horizontal axis represent a feature value, e.g. for one of the eight features described above. In the first graph the curve 301 gives the number of times (along the vertical axis) that an image part does not contain a vessel structure for a feature value derived from that image part in one the training images. Correspondingly, the curve 302 gives the number of times that an image part contains a vessel structure (e.g. a lumen 102) for a feature value derived from that image part in one of the training images. In the region of overlap of curves 301 and 302, the feature values are derived from image parts of which some did not contain a desired vessel structure and some did contain the desired vessel structure. At the intersection point, 50% of the image parts contained the desired vessel structure and 50% of the image parts of all training images did not contain the desired vessel structure. Thus, the feature value at the intersection point gives a 50% likelihood that an image part for a non-training image contains the desired vessel structure.

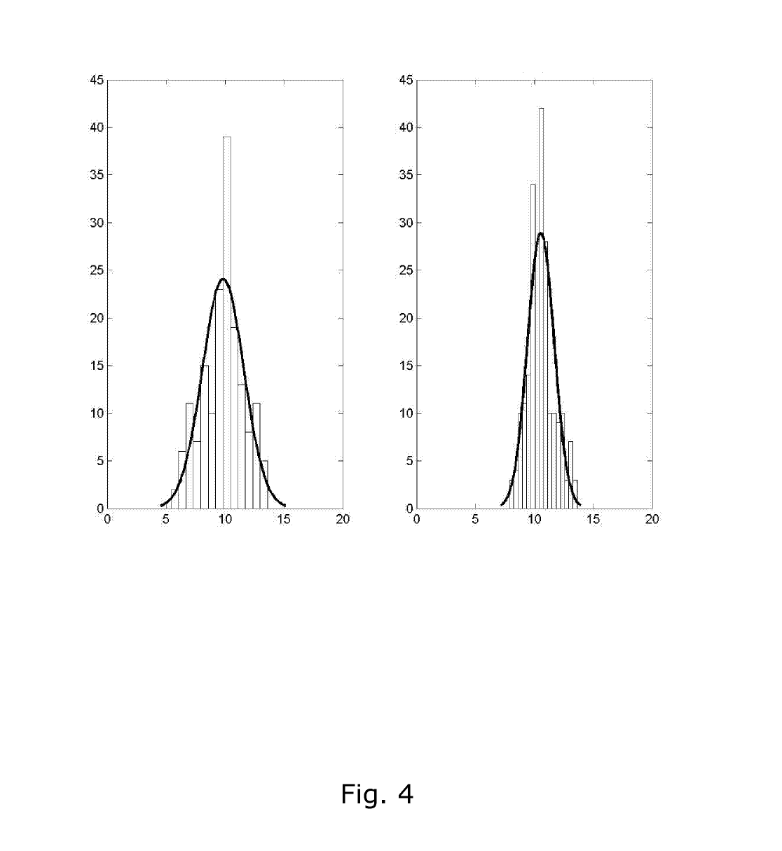

FIG. 4 shows how the curves 301 and 302 can be determined by forming histograms. Thus, the histogram to the left shows the number of times that a feature value was determined from an image part which did not contain the desired vessel structure, and the histogram to the right shows the number of times that a feature value was determined from an image part which did contain the desired vessel structure.

Probability distribution 303 in FIG. 3 gives the probability or likelihood that a feature value corresponds to an image part not containing the desired vessel structure. Probability distribution 304 in FIG. 3 gives the probability or likelihood that a feature value corresponds to an image part containing the desired vessel structure. The probability distributions 303, 304 are determined from curves 301, 302.

Accordingly, the probability distributions are determined from learning images by the steps:
determine image parts in each of the learning images by processing intensity values of the learning images,
determine the first feature value of each of one or more of the image parts in the learning images,
for each of the image parts,
   if an image part pictures a desired vessel structure insert the first feature value in a positive detection histogram, and
   if an image part does not picture the desired vessel structure insert the first feature value in a negative detection histogram,
from the positive and negative detection histograms, determine a probability distribution which describe the likelihood that an arbitrary image part from which an arbitrary first feature value is determined pictures a desired vessel structure,
repeat the steps by determine the second feature value of each of one or more of the image parts in the learning images.

In step 4 an adaptable closed circular contour for modeling e.g. the boundary between the vessel tissue 101 and the lumen 102 is created or defined as described in WO2012/107050 on page 16, line 32—page 17, line 9.

In step 5 the adaptable closed circular contour is deformed towards the boundary or wall between the vessel tissue 101 and the lumen 102 to obtain a mathematical model or description of the blood vessel structure, e.g. by use of an energy method as described in WO2012/107050 page 17, line 11—page 19, line 32.

In an embodiment in an optional step 6, the finally adapted contour from step 5 is used as an initial contour of the boundary between the vessel tissue and the lumen in an image part in a subsequent second medical image 100.

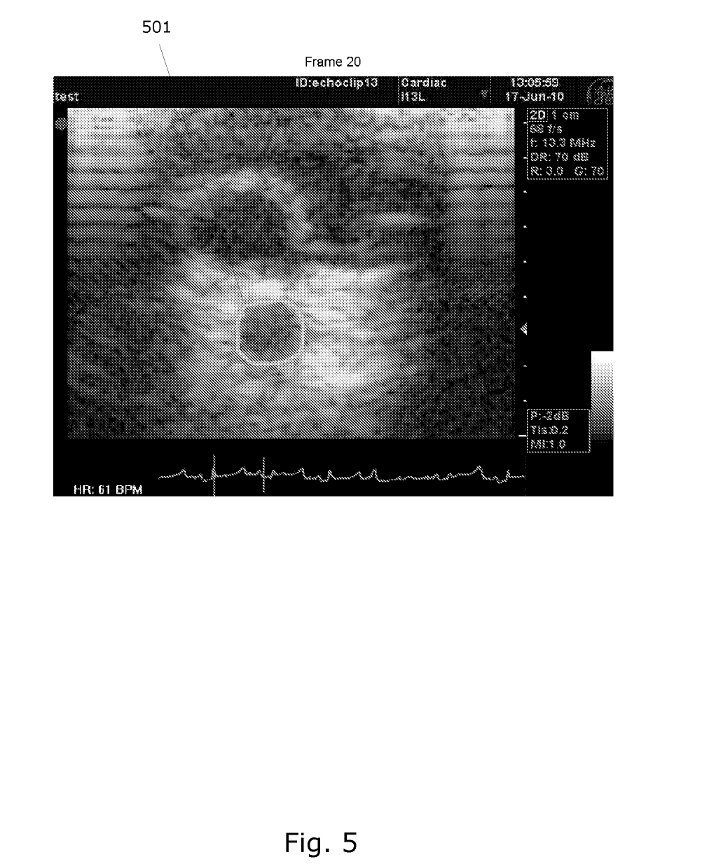

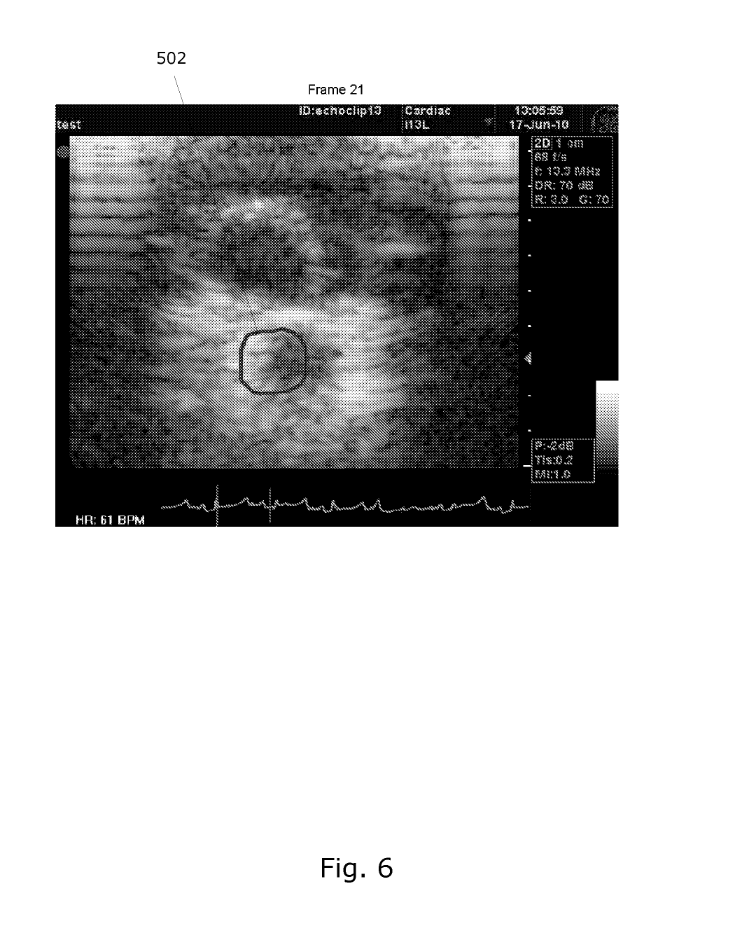

FIG. 5 shows a medical image (e.g. a first image in the time series of images) wherein a finally adapted contour 501 is formed. FIG. 6 shows a subsequent medical image in the time series wherein the finally adapted contour 501 is used as an initial contour 502 for the displaced blood vessel. In order to adapt or displace the initial contour 502 so that it models the lumen of the displace blood vessel the following steps are performed:
the geometric center of the contour 501 of the desired blood vessel structure in the first image is determined,
the finally adapted contour 501 is used as an initial contour 502 in a subsequent image in the time series of images,
an intensity center in the initial contour 502 is calculated from intensity values of pixels of the subsequent image which are contained within the initial contour 502 of the desired blood vessel structure in the first image,
the initial contour 502 is adapted or displaced so as to minimize the distance between the geometric center and the intensity center.

1. A system for detecting blood vessel structures in medical images obtained from a medical imaging device configured to obtain cross sectional views of blood vessels, where system comprises a processing unit configured for analysing the medical images by performing the steps:
determining image parts (113, 114, 213a, 213b, 214a, 214b) in a selected medical image (100), where the image parts are determined by processing intensity values of the image,
determining one or more feature values of each of one or more of the image parts, where the features values indicate if the image part from which the feature values are determined pictures a desired blood vessel structure,
based on the feature values, determine if the selected medical image shows the desired blood vessel structure.

2. A system according to claim 1, where the feature values are associated with a likelihood/probability that the image part from which the feature values are determined pictures a desired blood vessel structure.

3. A system according to claim 1, wherein the processing unit is configured for analysing the medical images by determining one or more of the following feature values of each of one or more of the image parts:
a) an intensity standard deviation feature value determined by calculating the standard deviation of intensity values of pixels contained in one of the image parts,
b) a mean intensity feature value determined by calculating the mean value of intensity values of pixels contained in one of the image parts,
c) a compactness feature value determined by calculating the boundary length and the area within the boundary of one of the image parts and comparing the boundary length with the area,
d) a vertical distance feature value determined by calculating a distance between a center value of one of the image parts and a center value of the medical image,
e) a horizontal distance feature value determined by calculating a distance between a center value of one of the image parts and a center value of the medical image,
f) a boundary gradient feature value determined by calculating the first derivative of the intensity values of pixels contained in one of the image parts,
g) an intensity variance feature value determined by calculating the variation of intensity values of pixels contained in one of the image parts, and
h) an aspect ratio feature value determined by calculating a major axis and a minor axis of one of the image parts and comparing the major axis with the minor axis.

4. A system according to claim 1, where processing unit is configured for analysing the medical images by performing the steps
determining first and second feature values of each of one or more of the image parts,
comparing the first and second feature values with respective first and second probability distributions, where the distributions describe the likelihood that the image part from which the feature values are determined pictures a desired blood vessel structure.
based on the comparison, determine if the selected medical image shows the desired blood vessel structure with a sufficiently high likelihood.

5. A system according to claim 4, wherein comparing the first and second feature values with respective first and second probability distributions comprises determining respective first and second probability values from the probability distributions corresponding to the feature values.

6. A system according to claim 5, further comprising calculating a sum of the first and second probability values.

7. A system according to claim 1, further comprising using the selected medical image for further image processing or discarding the selected medical image based on the determining if the selected medical image shows the desired blood vessel structure.

8. A system according to claim 1, wherein the medical images represent a time series of images showing a pulsating blood vessel, wherein the selected image is a first image in the time series of images, and wherein
the geometric center of a finally adapted contour (501) of the desired blood vessel structure in the first image is determined,
the finally adapted contour (501) is used as an initial contour (502) in a subsequent image (100) in the time series of images,
an intensity center in the initial contour (502) is calculated from intensity values of pixels of the subsequent image which are contained within the initial contour (502) of the desired blood vessel structure in the first image,
the initial contour (502) is adapted or displaced so as to minimize the distance between the geometric center and the intensity center.

9. A method for detecting blood vessel structures in medical images obtained from a medical imaging device configured to obtain cross sectional views of blood vessels, wherein the method comprises
determining image parts in a selected medical image, where the image parts are determined by processing intensity values of the image,
determining first and second feature values of each of one or more of the image parts,
comparing the first and second feature values with respective first and second probability distributions, where the distributions describe the likelihood that the image part from which the feature values are determined pictures a desired blood vessel structure.
based on the comparison, determine if the selected medical image shows the desired blood vessel structure with a sufficiently high likelihood.
